(19) United States
(12) Patent Application Publication
(10) Pub. No.: US 2025/0111906 A1
(43) Pub. Date: Apr. 3, 2025

(54) SYSTEMS AND METHODS FOR DETERMINGING AN ELECTROCHEMICAL SERIES FOR IONS IN INORGANIC SOLID MATERIALS

(71) Applicant: Toyota Research Institute, Inc., Los Altos, CA (US)

(72) Inventor: Timothy K. Mueller, Towson, MD (US)

(73) Assignees: Toyota Research Institute, Inc., Los Altos, CA (US); Toyota Jidosha Kabushiki Kaisha, Toyota-shi Aichi-ken (JP)

(21) Appl. No.: 18/650,185

(22) Filed: Apr. 30, 2024

Related U.S. Application Data
(60) Provisional application No. 63/587,195, filed on Oct. 2, 2023.

(51) Int. Cl. G16C 60/00; G16C 20/70

(52) U.S. Cl. CPC G16C 60/00; G16C 20/70

(57) ABSTRACT

A system includes a processor and a memory communicably coupled to the processor and storing machine-readable instructions that, when executed by the processor, cause the processor to calculate joint probability functions for electronic chemical potential and oxidation states for ion types of a solid inorganic material, calculate a likelihood score for a plurality of oxidation state sets for the individual ion types in the solid inorganic material, and select one set of oxidation state from the plurality of oxidation state sets for the individual ion types as a function of the likelihood score. In some variations, the joint probability functions are calculated with a trained machine learning module.

### CROSS-REFERENCE TO RELATED APPLICATIONS

This application claims the benefit of U.S. Provisional Application No. 63/587,195, filed Oct. 2, 2023, which is incorporated herein in its entirety by reference.

### TECHNICAL FIELD

The present disclosure relates generally to electrochemical properties of materials, and particularly to oxidation states of ions in solid materials.

### BACKGROUND

The standard reduction potential of an ion is a measure of the tendency of the ion to acquire electrons from or lose electrons to an electrode and thereby be reduced or oxidized, respectively. Also, the electrochemical series, broadly defined as a list of electrochemical reduction reactions sorted by standard reduction potential, is used to guide research in a variety of fields including biomaterials, materials synthesis, and energy storage, among others. The electrochemical series typically includes standard reduction potentials of different ions in aqueous solutions, which can be used to better understand the electrochemical behavior of ions in solid materials. However the efficacy of this approach, i.e., using the standard reduction potential of an ion in aqueous solution to understand the behavior of the ion in a solid material, is limited since some oxidation states (i.e., the nominal number of electrons that an atom either gains or loses) that exist in solid materials are not stable in aqueous solution, and the chemical environments of ions in the solid state can differ significantly from those of ions in aqueous solution.

The present disclosure addresses issues related to the electrochemical series and oxidation states of ions in solid-state materials (also referred to herein simply as “solid materials”), and other issues related to the electrochemical behavior or properties of solid-state materials.

### SUMMARY

This section provides a general summary of the disclosure and is not a comprehensive disclosure of its full scope or all of its features.

In one form of the present disclosure, a system includes a processor and a memory communicably coupled to the processor and storing machine-readable instructions that, when executed by the processor, cause the processor to calculate likelihood scores, based on joint probability functions for the electronic chemical potential and oxidation states for the species present in a solid inorganic material, for possible combinations of oxidation states in the material, and select one combination of oxidation states that is most likely to be observed in the material based on the likelihood score.

In another form of the present disclosure, a system includes a processor and a memory communicably coupled to the processor and storing machine-readable instructions that, when executed by the processor, cause the processor to train a machine learning module configured to calculate joint probability functions for the electronic chemical potential and oxidation states for species in a solid inorganic material. The memory also stores machine-readable instructions, that when executed by the processor, cause the processor to calculate the joint probability functions for the electronic chemical potential and oxidation states for species in a solid inorganic material, calculate a likelihood score for possible combinations of oxidation states for the species in the solid inorganic material, and select one combination of oxidation states that is most likely to be observed in the material based on the likelihood score.

In still another form of the present disclosure, a system includes a processor and a memory communicably coupled to the processor. The memory stores machine-readable instructions that, when executed by the processor, cause the processor to train a machine learning module configured to calculate joint probability functions for electronic chemical potential and oxidation states for species in a solid inorganic material with a training data set comprising integer, non-zero, oxidation states for species in inorganic materials with charge neutrality. The memory also stores machine-readable instructions that, when executed by the processor, cause the processor to calculate the joint probability functions for the electronic chemical potential and oxidation states for species in a solid inorganic material, calculate a likelihood score for a plurality of oxidation state sets for the individual ion types in the solid inorganic material, and select one set of oxidation states that is most likely to be observed in the material based on the likelihood score.

Further areas of applicability and various methods of enhancing the above technology will become apparent from the description provided herein. The description and specific examples in this summary are intended for purposes of illustration only and are not intended to limit the scope of the present disclosure.

### BRIEF DESCRIPTION OF THE DRAWINGS

The present teachings will become more fully understood from the detailed description and the accompanying drawings, wherein:

FIG. 1 shows a block diagram for a system for determining oxidation states for ion types in inorganic solid state materials according to the teachings of the present disclosure;

FIG. 2 shows a flow chart for a method for determining oxidation states for ion types in inorganic solid state materials according to the teachings of the present disclosure;

FIG. 3A shows a plot of probability functions for three oxidation states for a hypothetical cation A and boundary values ‘b’ consisting of b_{1+/2+}, b_{2+/3+}, b_{3+/4+}, and b_{4+/5+} according to the teachings of the present disclosure;

FIG. 3B shows a plot of the probability functions from FIG. 3A with the step functions at the boundaries replaced by logistic functions and the set of midpoint values of the logistic functions ‘b’, the same as the set of midpoint values of the boundary values ‘b’ from FIG. 3A according to the teachings of the present disclosure;

FIG. 3C shows a plot of compact representation of the probability functions in FIG. 3B according to the teachings of the present disclosure;

FIG. 3D shows the plot in FIG. 3C with an additional plot of the probability functions for a hypothetical anion X according to the teachings of the present disclosure;

FIG. 3E shows a plot of set of ions that maintain charge neutrality for a material with composition AX₂ according to the teachings of the present disclosure;

FIG. 3F shows a plot of another set of ions that maintain charge neutrality for a material with composition AX₂ according to the teachings of the present disclosure;

FIG. 4 shows a plot of standard reduction potentials for twenty-five (25) redox couples in aqueous solution vs. the ICSD-derived reduction potential” (IRP) according to the teachings of the present disclosure with five redox couples highlighted as an illustrative example;

FIG. 5A shows graphical representations for boundaries for twenty-four (24) elements of the periodic table according to the teachings of the present disclosure;

FIG. 5B shows graphical representations for boundaries for selected polyatomic anions as well as their corresponding monatomic cations;

FIG. 6A shows a plot of oxidation state prediction accuracy for five (5) different methods;

FIG. 6B shows a plot of oxidation state ranges for Mn, Ni, S, and O with logistic functions for clarity; and

FIG. 6C shows a plot of the relationship between the likelihood score of a composition and the probability that a previous search for materials found a material with that composition on the thermodynamic convex hull.

### DETAILED DESCRIPTION

The present disclosure provides systems and methods for predicting the oxidation state(s) of ion types of inorganic solid materials. As used herein, the term “ion” refers to an atom or group of atoms that have a non-zero oxidation state, the phrase “ion types” refers to an atom or a group of atoms that become(s) an ion when in a non-zero oxidation state, and the phrase “oxidation state” refers to an integer value that is equal to the total number of electrons removed or added from an atom or a group of atoms, and/or transferred from an atom or group of atoms to another atom or group of atoms, to reach a present state.

The systems and methods according to the teachings of the present disclosure calculate joint probability functions for electronic chemical potential and oxidation states for ion types using machine learning. The systems include a machine learning module or algorithm trained with non-zero, oxidation states for ion types of inorganic solid materials having charge neutrality. In some variations, the systems include a machine learning module or algorithm trained with experimentally determined non-zero oxidation states for ion types of inorganic solid materials having charge neutrality. As a result, a graphical representation of the oxidation states, as a function of electronic chemical potential, for ion types that form inorganic solid materials is provided. In addition, and after the joint probability functions for electronic chemical potential and oxidation states for the ion types have been calculated, one or more likelihood scores for different oxidation state combinations for sets of individual ion types forming a given or particular inorganic solid material is/are calculated, and one set of oxidations states for the ion types (i.e., an oxidation state for each ion) is selected as a function of the one or more likelihood scores, e.g., as a function of a highest likelihood score. In some variations, and in order to ensure charge neutrality, up to one ion type in a given inorganic material is allowed to have two different oxidation states.

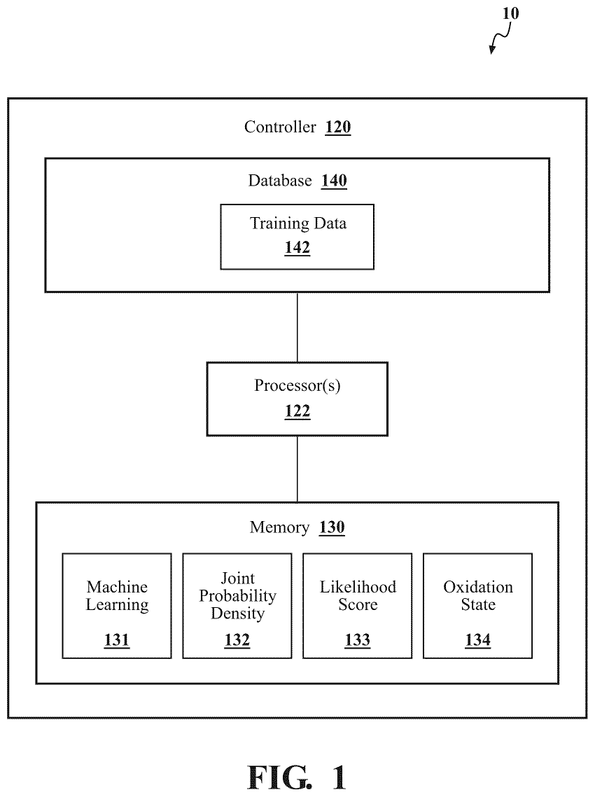

Referring to FIG. 1, a block diagram for a system 10 according to one form of the present disclosure is shown. The system includes a controller 120 with a processor 122, a memory 130, and a database 140. The processor 122 may be a part of the controller 120, the controller 120 may include a separate processor from the processor 122 of system 10, or the controller 120 may access the processor 122 through a data bus or another communication path.

The memory 130 stores a machine learning module 131, a joint probability density module 132, a likelihood score module 133, and an oxidation state module 134 (collectively referred to herein as the “likelihood model”) such that the memory 130 provides for estimation and/or determination of the oxidation state for individual ion types that form an inorganic solid material as described in greater detail below. The memory 130 can be constructed as a random-access memory (RAM), read-only memory (ROM), a hard-disk drive, a flash memory, or other suitable memory for storing the machine learning module 131, joint probability density module 132, likelihood score module 133, and oxidation state module 134. The machine learning module 131, joint probability density module 132, likelihood score module 133, and oxidation state module 134, for example, can be constructed as computer-readable instructions that when executed by the processor 122 cause the processor 122 to perform the various functions disclosed herein.

The database 140 stores, among other things, training data 142 (e.g., experimentally and/or non-experimentally determined, non-zero oxidation states for ion types of inorganic solid materials with charge neutrality). In some variations, the training data 142 includes oxidations states for ion types provided by the Inorganic Crystal Structure Database (ICSD) available at https://icsd.products.fiz-karlsruhe.de/. The database 140 is constructed as an electronic data structure stored in the memory 130 or another data store, such as a cloud-based storage, a removable memory device, or another suitable location that is accessible to the machine learning module 131, joint probability density module 132, likelihood score module 133, and oxidation state module 134. The database 140 is configured with routines that can be executed by the processor 122 for analyzing stored data, providing stored data, organizing stored data, and so on. And in at least one variation, the database 140 stores data described above (as well as other data) the machine learning module 131, joint probability density module 132, likelihood score module 133, and oxidation state module 134 to execute various functions.

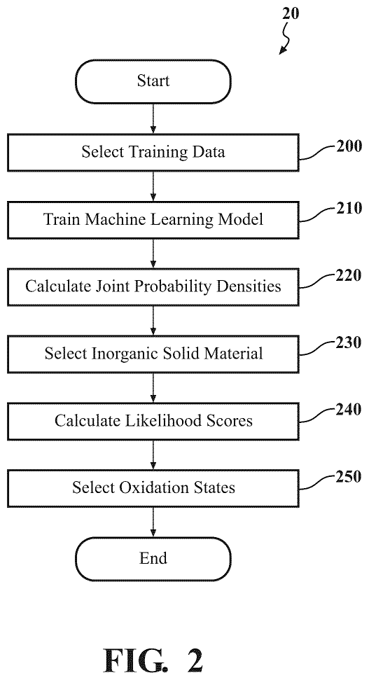

Referring to FIG. 2, a method 20 according to one form of the present disclosure is shown. The method 20 includes selecting oxidation state training data at 200, e.g., experimentally and/or non-experimentally determined, non-zero, oxidation states for ion types of inorganic solid materials having charge neutrality, and training a machine learning model as discussed in greater detail below at 210.

The method 20 also includes calculating joint probability functions for electronic chemical potential and oxidation state for ion types that form inorganic solid materials at 220, selecting an inorganic solid material at 230, and calculating one or more likelihood scores for different oxidations states of the ion types that form the inorganic solid material at 240. Then, the method 20 selects an oxidation state for each of the ion types that form the inorganic solid material at 250.

Not being bound by theory, the system 10 and method 20 assume the oxidation state of an ion is determined by both the electronic chemical potential (μₑ) and the atomic structure of its environment, i.e., the environment of an ion in a given inorganic solid material. For example, consider a hypothetical cation ‘A’ that can be found in the +2, +3, and +4 oxidation states. It should be understood that there will be a range of electronic chemical potential values at which the cation A is stable in a +3 oxidation state, another range of electronic chemical potential values at which the cation A is stable in a +2 oxidation state, and still another range of electronic chemical potential values at which the cation A is stable in a +4 oxidation state. In addition, and assuming the electronic chemical potential values are such that the cation A is stable in the +3 oxidation state, as the electronic chemical potential becomes more reducing, the +2 oxidation state will eventually become energetically favorable. That is, the cation A will undergo a transition from the +3 oxidation state to the +2 oxidation state at a given electronic chemical potential. Similarly, at more oxidizing potentials the cation A in the +3 oxidation state will be oxidized to a +4 oxidation state. Accordingly, there are “boundaries” between the different oxidation states and these boundaries are defined herein as the electronic chemical potential values at which a transition between oxidation states occurs. And for each element, the boundaries between oxidation states will be at different electrical chemical potential values.

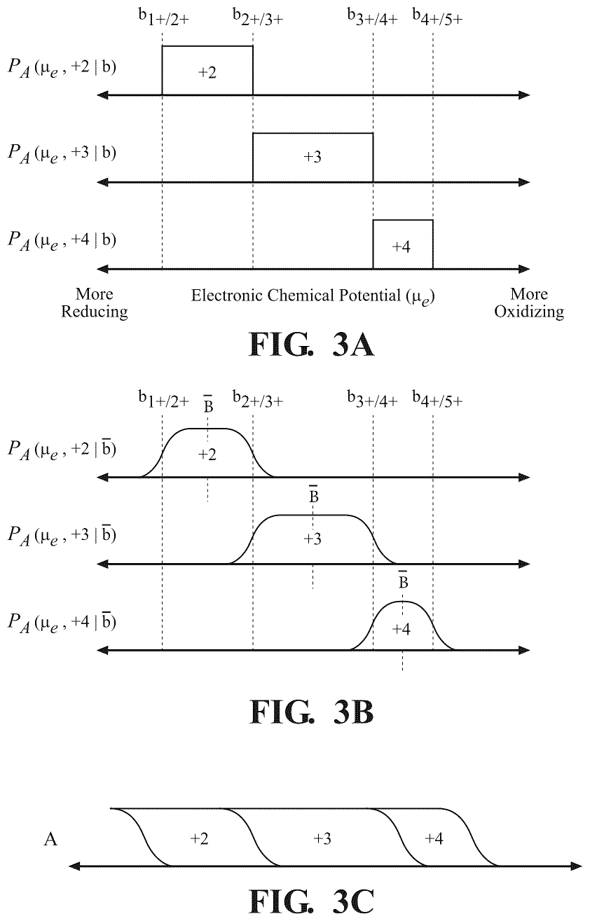

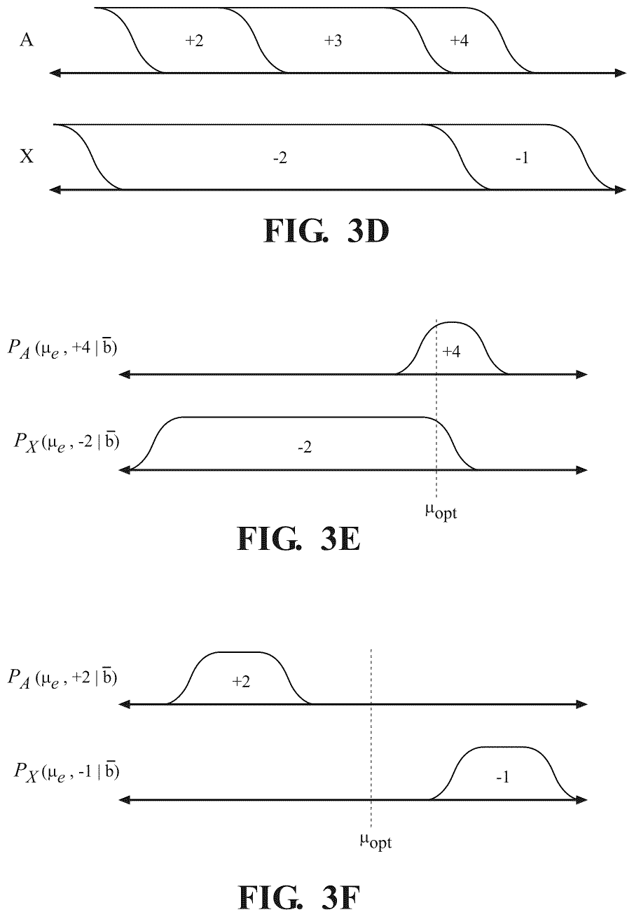

Mathematically, the system 10 and method 20 define ‘B’ as the set of all boundaries between oxidation states, ‘S’ as the set of all allowed oxidation states for an ion type, and a joint probability density for the electronic chemical potential μₑ and a given oxidation state sⱼ conditional on B as Pₘ(μₑ, sⱼ|B) where is ‘m’ represents a given ion type. For example, and with reference to FIG. 3A, the joint probability functions for the +2, +3, and +4 oxidation states as a function of the electronic chemical potential μₑ for the cation A (i.e., m=A) are shown. For convenience, the system 10 and method 20 do not normalize the joint probability functions, but rather assigns them a value of one (1) when the electronic chemical potential μₑ is between the boundaries for a given oxidation state sⱼ, and zero (0) when the electronic chemical potential is outside the boundaries for the given oxidation state sⱼ.

It should be understood that the electronic chemical potential boundary between oxidation states will depend on the local atomic environment. Accordingly, and with reference to FIG. 3B, the system 10 accounts for the uncertainty of the local atomic environment by replacing the step functions in Pₘ(μₑ, sⱼ|B) with optional smooth logistic functions centered at boundary midpoints, B̅, and for visualization, arranging the joint probability functions, replaced with the smooth logistic functions, on a single horizontal axis provides a graphical representation of the oxidation states as a function of electronic chemical potential as illustrated in FIG. 3C. In some variations, the system 10 accounts for the uncertainty of the local atomic environment by replacing the step functions in Pₘ (μₑ, sⱼ|B) with optional smooth logistic functions of fixed width centered at boundary midpoints, B̅.

Regarding predicting or determining an oxidation state for each ion type ‘m’ that forms an inorganic solid material, the system 10 and method 20 determine the joint probability functions for electronic chemical potential and oxidation state of each ion as graphically illustrated in FIG. 3D for an inorganic solid material formed from the cation A and an anion X. In addition, and using the principle that all ion types in an inorganic solid material experience or have the same electronic chemical potential, an expression for the relative likelihood of observing a set of ions, I, at a given electronic chemical potential μₑ in the inorganic solid material is:

$$P ( {μ}_{e} I \vert \overline{B} ) = {\prod }_{{m}^{{s}_{j}} ∈ I} {P}_{m} ( {μ}_{e} , {s}_{j} \vert \overline{B} ) \qquad ( 1 )$$

It should be understood that since Pₘ (μₑ, sⱼ|B̅) is defined such that P(μₑ, I|B̅) ranges from 0 to 1, P(μₑ, I|B̅) is not normalized, and hence is not strictly a probability density. Accordingly, P(μₑ, I|B̅) is referred to herein as a “likelihood score”. In some variations, the likelihood score is calculated via an intermediate step and the expression:

$${P}_{m} ( {μ}_{e} {s}_{j} \vert \overline{B} ) = min ( ( \frac{1}{1 + exp ( \overline{b} {e}_{min}} ) , ( \frac{1}{1 + exp ( {μ}_{e} - {\overline{b}}_{max}} ) ) \qquad ( 2 )$$

where the “min” function returns the minimum value of the two arguments, the “exp” function is the natural exponential function, b̅ₘᵢₙ is the lower chemical potential boundary midpoint for oxidation state sⱼ, and b̅ₘₐₓ is the upper boundary midpoint for oxidation state sⱼ. Then, defining the electronic chemical potential μₒₚₜ as the value of the electronic chemical potential μₑ that maximizes the likelihood score for a given set of ion types (i.e. the most likely electronic chemical potential for the material), the system 10 and method 20 define the likelihood score:

$${P}_{opt} ( I \vert \overline{B} ) = P ( {μ}_{eopt} , I \vert \overline{B} ) = {\prod }_{{m}^{{s}_{j}} ∈ I} {P}_{m} ( {μ}_{opt} , {s}_{j} \vert \overline{B} ) \qquad ( 3 )$$

as an estimate for the oxidation states of individual ion types that form an inorganic solid material. Accordingly, the system 10 and method 20 estimate a likelihood score for a combination of oxidation states most likely to occur in an inorganic solid material by identifying the combination that maximizes Pₒₚₜ(I|B̅).

For example, and with reference to FIGS. 3E and 3F, and the assumption that the inorganic solid material is AX₂, FIG. 3E illustrates one combination of oxidations states for A and X (i.e., A has an oxidation state of +4 and X has an oxidation state of −2), while FIG. 3F illustrates another combination of oxidation states for A and X (i.e., A has an oxidation state of +2 and X has an oxidation state of −1). Both combinations provide for the AX₂ inorganic solid material having charge neutrality, however, the A⁴⁺ and X²⁻ oxidation states have a higher likelihood score since there is a single electronic chemical potential, μₒₚₜ, at which the 4+ and 2− oxidation states are both likely to be observed as illustrated in FIG. 3E.

### Training the Machine Learning Model

Regarding training the machine learning model, in some variations the machine learning module 131 is parameterized by the midpoints of the boundaries ‘B̅’ between oxidation states sⱼ. And in at least one variation, the machine learning module 131 learns the parameters by maximizing the geometric mean of Pₒₚₜ(I|B̅) over a set of training data in the form of experimentally observed inorganic materials with ion types labeled with integer, non-zero oxidation states such that charge neutrality is maintained. Particularly, the set of training data can originate from the ICSD. However, in at least one variation data entries containing rare oxidation states, defined as fewer than 25 entries in the data set, are removed.

In some variations, the machine learning module 131 is parameterized with an added regularization term to prevent the minimum and maximum boundaries from drifting to infinity. For example, in at least one variation the fit of the minimum and maximum boundaries is regularized with a term that penalizes the difference between the values of the minimum and maximum boundaries. And in some variations, this term is included in an objective function represented by the expression:

$$O ( \overline{B} \vert I ) = {\sum }_{I ∈ T} ln ( {L}_{opt} ( \overline{B} \vert I ) ) - λ {\sum }_{m} ( {\overline{b}}_{max . m} - {\overline{b}}_{min , m} ) \qquad ( 4 )$$

where the sum Σ(Lₒₚₜ(B̅|I)) is over all entries in a training set T, the sum Σ_{M}(b̅_{max.M}−b̅_{min,M})
is over all species (elements and polyatomic clusters) in the machine learning model, b̅_{max.M} is the maximum boundary midpoint for material component m, b̅_{min,M} is the corresponding minimum boundary midpoint, and λ is a regularization parameter. In at least one variation, the regularization parameter λ had a value of 5×10⁻⁶, as it constrained the boundaries with negligible impact on the calculated likelihoods. It should be understood that since the maximum boundary and the minimum boundary can be determined in part by minimizing the difference between the two boundaries, the minimum and maximum boundaries calculated this way do not necessarily reflect the full electronic chemical potential range at which the most reduced and most oxidized states may be stable.

The constraint that the oxidation states must increase monotonically as the electronic chemical potential becomes more oxidizing is reinforced by parameterizing the differences between successive boundary midpoints and squaring the parameters to ensure these differences were always non-negative. In addition, to maximize O(B̅|I) a conjugate gradient algorithm was used and ten different machine learning models were trained simultaneously, with each training run initialized with different random values for B. Slight variation in the optimized value of O(B̅|I) was found (with a standard deviation of 2×10⁻⁵) or in the final parameter values over the ten models, thereby suggesting this approach consistently found parameters close to the global optimum. And naturally, the machine learning model with the maximum value of O(B̅|I) was selected for subsequent use.

It should also be understood that oxidation states in the ICSD are assigned through a combination of human labeling and automated procedure(s), and such assignment of oxidation states can introduce errors into the data that propagate to errors in the machine learning module 131. Accordingly, in some variations, entries for which the most likely oxidation states predicted by the machine learning module 131 can be different than the most likely oxidation states provided or listed in the ICSD. And in an effort to quantitate the difference of confidence of the predicted oxidation states, a global instability index (GII) defined as the root-mean-square difference between the oxidation state of each atom and the bond valence sum on that atom was calculated. And in instance where the predicted oxidation states provided by the machine learning module 131 results in a lower GII than the oxidation states in the ICSD, the atom or molecular species is removed from the training data 142. And as such, oxidation states are assigned to sites such that the oxidation state monotonically increased with the bond valence sum, as this assignment minimized the GII.

In some variations, the bond valence values are calculated using parameters published in the reference article “Atom Sizes and Bond Lengths in Molecules and Crystals”, O'Keeffe et al., J. Am. Chem. Soc. 113, 3226-3229, which is incorporated herein by reference. Accordingly, and as disclosed by the O'Keeffe et al. publication, bond valences are only calculated between two atoms if at least one of them was drawn from the following list of allowed anions: H, B, C, Si, N, P, As, Sb, O, S, Se, Te, F, Cl, Br, I, and Ge. In addition, Bi can be added to the list of anions, as it can have negative oxidation states in some inorganic solid materials. Also, if both bound atoms (ions) for a molecule are on or in the list of allowed anions noted above, whichever has the lowest Allred-Rochow electronegativity as taught in the reference articles “A scale of electronegativity based on electrostatic force”, Allred et al., Journal of Inorganic and Nuclear Chemistry 5, 264-268, and “A complete table of electronegativities”, Little et al., Journal of Chemical Education 37, 231, can be considered to be the anion.

In some variations, and if the Allred-Rochow electronegativities are the same, or unavailable, then the Pauling electronegativities as disclosed in the reference articles “THE NATURE OF THE CHEMICAL BOND. IV. THE ENERGY OF SINGLE BONDS AND THE RELATIVE ELECTRONEGATIVITY OF ATOMS, Pauling, J. Am. Chem. Soc. 54, 3570-3582, and CRC Handbook of Chemistry and Physics Vol. 88th Edition (Internet Version 2008) (ed David R. Lide) (CRC Press/Taylor & Francis, 2008) are used. Also, atoms are only considered to be bonded (with non-zero bond valence) if the distance between atoms in Angstroms was no more than rᵢ+rⱼ+1.25, where rᵢ and rⱼ are parameters for each atom from reference 25. If bond valence parameters are not available for some elements in the material, or if any bond valence sum in the material is equal to zero, the GII was not calculated.

It should be understood that, in some variations, if the most likely oxidation states predicted by the machine learning module 131 resulted in a lower GII than the oxidation states listed or provided in the ICSD, it meant the machine learning module 131 had discovered or determined oxidation states that were better than those in the ICSD from the perspectives of both electronic structure (according to the machine learning module 131) and atomic structure (according to the bond valence sums). As this gave low confidence that the ICSD oxidation state(s) were correct, such entries were removed from the training set, and the model was then retrained. This procedure was repeated until no more entries met our criterion for removal. For example, data entries that were removed ranged from those that were likely to be incorrect in the ICSD (such as La²⁺₂Ni⁴⁺O²⁻₄), to those that were “close calls.” An example of the “close call” type is CuTe, which is labeled as Cu²⁺Te²⁻ according to the ICSD, and is sometimes known as copper (II) telluride.¹⁰ However, the machine learning module 131 predicted a slightly higher likelihood score for Cu¹⁺Te¹⁻, even when all of the Cu²⁺Te²⁺ entries are included in the training set. The atomic structure of the material, with alternating layers of Cu and Te atoms, as well as experimental evidence indicating that Cu is in an oxidation state close to +1 in this material, suggest that Cu¹⁺Te¹⁻ may be a more accurate set of oxidation states.

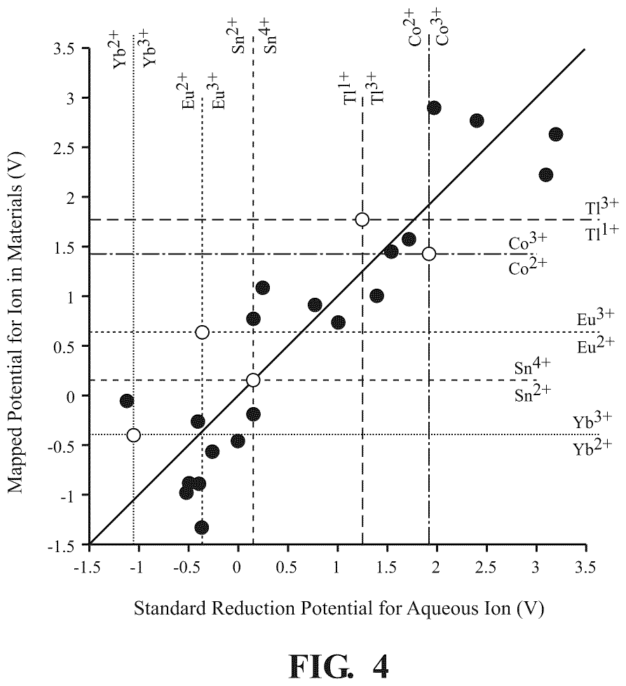

As the model was trained on a set of compositions with labeled oxidation states, it contains no information about energies or electronic chemical potentials. Thus the training procedure does not provide physically meaningful units for the “electronic chemical potential” axis. All that is known about this axis is that it is a monotonically increasing function of the true electronic chemical potential. Accordingly, and to assign values to electronic chemical potential axis that are somewhat physically meaningful, the boundary midpoint values predicted by the machine learning module 131 were compared to standard reduction potentials of ions in aqueous solution. Particularly, 25 such redox pairs that exist both in our model and in the electrochemical series data from the CRC Handbook of Chemistry and Physics were identified. And as the relationship between the standard reduction potentials and the machine-learned boundary values is roughly linear (with a Pearson correlation coefficient of 0.89), a linear map between the two sets of values was developed as shown in FIG. 4. This map allows us to express the electronic chemical potential in terms of ICSD-derived reduction potential (IRP) values (Eₘₐₚ), with units that approximately correspond to volts. However, it should be understood that other comparisons of the boundary midpoint values predicted by the machine learning module 131 (e.g., comparison to electronic densities of states) can be used to assign values to electronic chemical potential axis that are somewhat physically meaningful.

Particularly, the IRP was calculated using the following expression:

$${E}_{m a p} = ( {μ}_{e} - {\overline{μ}}_{e} ) \frac{‖ E - \overline{E} ‖}{‖ {μ}_{e} - {\overline{μ}}_{e} ‖} + \overline{E} \qquad ( 5 )$$

where Eₘₐₚ is the IRP, μₑ is the electrochemical potential in the units used to train the model, E is the set of standard reduction potentials used for the fit, μₑ is the corresponding set of boundary midpoint values, and Ē and μ̅ are vectors in which every element is the mean value of E and He, respectively. Here ∥x∥ represents the l² norm of x. The mapping in equation (5) is equivalent to standardizing each of the data sets (the standard reduction potentials and the boundary midpoints) so that they have a mean of zero and standard deviation of 1, performing a principal component analysis on the standardized data, and then transforming the first component back to the original scale of the standard reduction potentials. This approach ensures a linear fit in a way that does not depend on which data set is considered the dependent variable.

As observed in FIG. 4, the ordering of the redox couples predicted by the machine learning module 131 (horizontal lines corresponding to the vertical axis) differs from that of the solvated ions in the standard electrochemical series (vertical lines corresponding to the horizontal axis). This is primarily due to the fact that the machine learning module 131 was trained on tens of thousands of data points in solid state materials, whereas the data points for solvated ions are representative of one particular liquid environment. The change in ordering is highlighted for five redox couples shown in FIG. 4. The standard reduction potentials for these couples are ordered as Yb^{2+/3+}<Eu^{2+/3+}<Sn^{2+/4+}<Tl^{1+/3+}<Co^{2+/3+}, whereas the boundary midpoints in our model are ordered as Yb^{2+/3+}<Sn²⁺⁴⁺<Eu^{2+/3+}<Co^{2+/3+}<T^{1+/3+}. A comparison of the Sn^{2+/4+} and Eu^{2+/3+} redox couples provides an illustrative example. Based on the standard reduction potentials, there should be a potential range at which Eu³⁺ and Sn²⁺ coexist. However based on the predictions of the machine learning module 131, it is more likely for Sn⁴⁺ and Eu²⁺ to coexist. In addition, the predictions of the machine learning module 131 are more consistent with the ICSD data, in which there are seven entries (representing five distinct compositions) in which Sn⁴⁺ and Eu²⁺ coexist, and none in which Eu³⁺ and Sn²⁺ coexist.

Table 1 below contains all redox couples for which a similar analysis was performed. In most cases, the prediction of the machine learning module 131 was in better agreement with the ICSD data, which is expected as this data was used to train the machine learning module 131. A notable exception occurs when one of the redox couples is H¹⁻/H¹⁺, for which the data in the ICSD is more likely to be inconsistent with itself, and the electrochemical series is more consistent with the ICSD data in a relatively sizable percentage of cases. In organic groups such as formate (CHO₂)⁻ or methylammonium (CH₃—NH₃)⁺, it is conventional to assign hydrogen a +1 oxidation state, which is also generally predicted by our model. However this results in neighboring atoms with either positive oxidation states (as in formate) or negative oxidation states (as in methylammonium), which is counter to the expectation in inorganic chemistry that anions should be next to cations, and vice versa. This may lead to inconsistent labeling of hydrogen oxidation states in the ICSD.

TABLE 1

| Couple | Couple | ΔE_{aq} | ΔEₘₐₚ | | |
|---|---|---|---|---|---|
| 1 | 2 | (V) | (V) | #E_{aq} | #Eₘₐₚ |
| In^{2+/3+} | Yb^{2+/3+} | −0.56 | 0.49 | 0 | 3 |
| Ge^{2+/4+} | Yb^{2+/3+} | −1.05 | 0.06 | 0 | 2 |
| Ti^{2+/3+} | Cr^{2+/3+} | −0.04 | 1.07 | 0 | 1 |
| V^{2+/3+} | Cr^{2+/3+} | −0.15 | 0.30 | 0 | 1 |
| Ge^{2+/4+} | Cr^{2+/3+} | −0.41 | 0.20 | 0 | 17 |
| U^{3+/4+} | H^{1-/1+} | −0.60 | 0.93 | 2 | 6 |
| In^{2+/3+} | H^{1-/1+} | −0.63 | 0.84 | 0 | 1 |
| V^{2+/3+} | H^{1-/1+} | −0.86 | 0.51 | 4 | 3 |
| Ge^{2+/4+} | H^{1-/1+} | −1.12 | 0.41 | 0 | 3 |
| Yb^{2+/3+} | H^{1-/1+} | −0.06 | 0.35 | 2 | 4 |
| Cr^{2+/3+} | H^{1-/1+} | −0.71 | 0.21 | 8 | 1 |
| Np^{3+/4+} | H^{1-/1+} | −1.26 | 0.13 | 0 | 2 |
| Ge^{2+/4+} | Eu^{2+/3+} | −0.36 | 1.10 | 0 | 16 |
| Sn^{2+/4+} | Eu^{2+/3+} | −0.51 | 0.49 | 0 | 7 |
| Co^{2+/3+} | Ce^{3+/4+} | −0.20 | 0.15 | 5 | 0 |
| Au^{1+/3+} | Tl^{1+/3+} | −0.15 | 0.76 | 0 | 1 |
| Mn^{2+/3+} | Tl^{1+/3+} | −0.29 | 0.31 | 0 | 4 |
| Cu^{2+/3+} | Ag^{1+/2+} | −0.42 | 0.12 | 1 | 2 |

### Examples

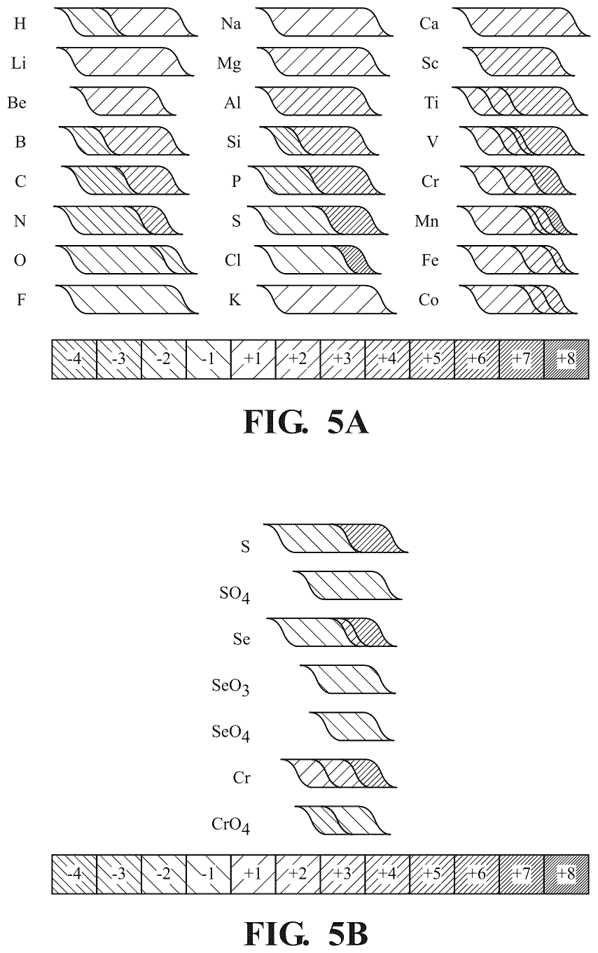

As noted above, the system 10 and method 20 provide for calculating joint probability functions for electronic chemical potential and oxidation state for ion types that form inorganic solid materials. For example, and with reference to FIGS. 5A and 5B, the system 10, after training of the machine learning module 131, calculated joint probability functions for electronic chemical potential and oxidation states for the first twenty-seven (27) elements in the periodic table excluding noble gases (FIG. 5A), and selected polyatomic anions with their corresponding monatomic cations (FIG. 5B). The oxidation state key is shown below the calculated joint probability functions in FIGS. 5A and 5B.

The trends in the fitted boundaries shown in FIGS. 5A-5B are broadly consistent with what would be expected. For example, the widths of the electronic chemical potential ranges at which anions are stable become wider as electronegativity increases. The model also explains some previously observed patterns, such as the tendency for electropositive anions to appear with metal ions with low oxidation states. Also, it should be understood that ranges for some oxidation states, such as Cr⁴⁺ and Cr⁵⁺, are too narrow to be observed in FIGS. 5A-5B, suggesting that such states may only rarely become more stable than a mixture of the more common states on either side of them.

One application of the system 10 and/or method 20 is the prediction of oxidation states from a material composition. Prior knowledge of a material's oxidation states can be useful for predicting material properties, e.g. by featurizing a machine learning model. Oxidation state prediction is often done using the bond valence model, which requires prior knowledge of the atomic structure. Other structure-based methods have been developed to predict oxidation states for metal-organic frameworks, metal ion types coordinated by oxygen, and metal sites in coordination complexes. For example, a tool called BERTOS directly predicts oxidation states from a material composition using a neural network transformer model (see Fu, N. et al. Composition Based Oxidation State Prediction of Materials Using Deep Learning Language Models. Advanced Science, 2301011.

In an effort to judge or score the performance of the predictions form the machine learning module 131, such predictions were compared with predictions from BERTOS, an algorithm used by PyMatGen based on the bond valence model as disclosed in the reference article Python Materials Genomics (pymatgen): A robust, open-source python library for materials analysis. Computational Materials Science, Ong, S. P. et al., 68, 314-319, (2013), and a baseline “frequency model” that depends only on the frequency at which each oxidation state appears in the ICSD.

For the frequency model, a frequency score for an ion is proportional to the number of times an ion appears in a data set and normalized so that the sum of all frequency scores for each element is 1. The frequency score for a material is simply the product of the frequency scores for the distinct ions in the material. Accordingly, the frequency score can and did select oxidation states similar to how in the likelihood score can and did select oxidation states, and these two scores can and were evaluated using ten-fold cross-validation to ensure a model was never evaluated on a composition on which it was trained.

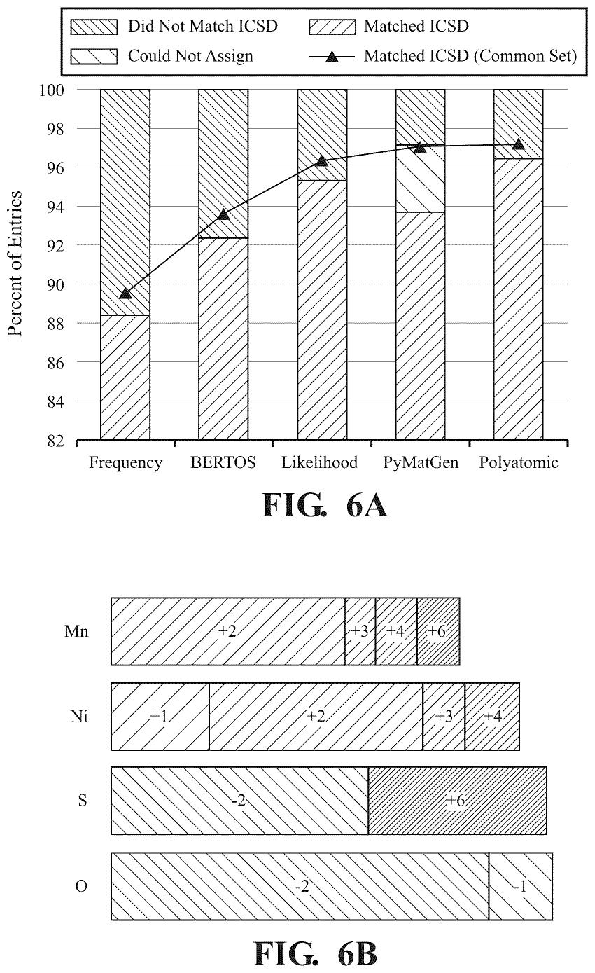

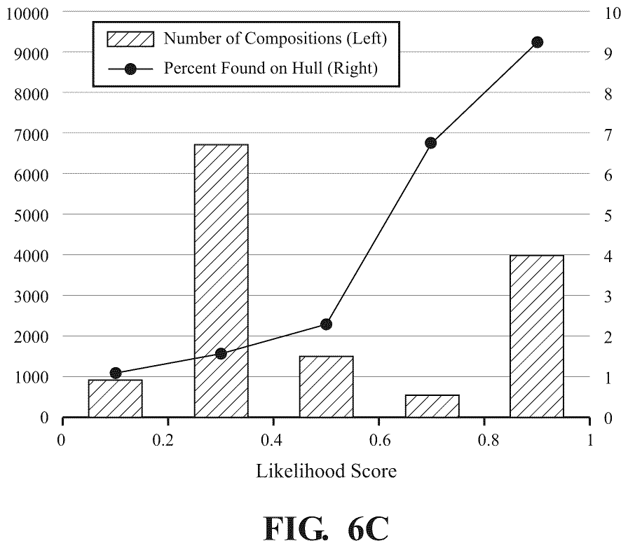

The performance of each model, i.e., the likelihood model of the present disclosure, the BERTOS model, the PyMatGen model, and the frequency score model, was based or evaluated on the fraction of entries for which all assigned oxidation states agree with the ICSD. To compare the models a subset of 53,246 entries that are covered by the chemical spaces of all evaluated models was used. The PyMatGen model and (to a much lesser extent) BERTOS model were unable to calculate oxidation states for all entries in this subset of 53,246 entries as shown in FIG. 6A, so an additional common set of 51,408 entries was constructed for which all models were able to successfully assign oxidation states. On this constructed set, the BERTOS model performed better than the frequency score model, and the likelihood score model performed better than the BERTOS model. The PyMatGen model and the likelihood score model based on labeled polyatomic ion types performed the best and were able to assign oxidation states in agreement with the ICSD for 97.0 and 97.2 percent of the entries, respectively. To evaluate the extent to which the cleaning procedure biased the results in favor of the likelihood score model, the same analysis on the uncleaned data set was performed agreement with the ICSD dropped the most for the frequency model and least for BERTOS model, but the relative performance between models was similar to that shown in FIG. 6A.

In addition to the above, it should be understood that ordering of the boundary midpoint values with respect to the IRP provides useful information for materials electrochemists. For example, the stability of nickel-based cathode materials in lithium-ion batteries can be improved by substituting Mn for Ni³⁺. Rather than creating a mixture of Mn³⁺ and Ni³⁺, Mn oxidizes to Mn⁴⁺ and reduces the Ni³⁺ to Ni²⁺, a more stable ion. The boundary midpoint values in our model make it clear at a glance that this is the expected behavior as illustrated in FIG. 6B (illustrated without smooth logistic functions), as there is no electronic chemical potential at which Mn³⁺ and Ni³⁺ are likely to coexist. Similarly, it is apparent why high-voltage batteries are generally oxides rather than sulfides, as S²⁻ gets oxidized at an IRP that is too low.

Not being bound by theory, it should be understood that the likelihood score model of the present disclosure can and does facilitate structure prediction since some ions are commonly found in certain local environments. For example, O⁻ is almost always found in a peroxide ion. Thus knowledge about the oxidation state of O gives insight into whether a material is an oxide or a peroxide. More generally, the ability to accurately determine oxidation states from composition makes it possible to reverse the usual application of the bond valence model; rather than determining oxidation states from structure, it becomes possible to use knowledge of the oxidation states to inform a structure search, e.g. by prioritizing structures that have low global instability indices.

It should also be understood that are also potential applications for the likelihood score itself. For example, using a set of structures generated in a previous materials discovery project (see Ye, W., Lei, X., Aykol, M. & Montoya, J. H. Novel inorganic crystal structures predicted using autonomous simulation agents. Scientific Data 9, 302, (2022), compositions that have a low likelihood score were found to be relatively unlikely to be found on the thermodynamic convex hull as shown in FIG. 6C.

It should be understood from the teachings of the present disclosure that systems and methods that predict oxidation states for ion types in an inorganic solid material, and based only on the composition of the inorganic solid material are provided. The systems and methods use a machine learning model trained on experimentally and/or non-experimentally determined, non-zero oxidation states for ion types of inorganic solid materials having charge neutrality. In some variations, the systems and methods provide a graphical representation of the oxidation states, as a function of electronic chemical potential, for ion types that form inorganic solid materials. In addition, and after joint probability functions for electronic chemical potential and oxidation state for the ion types have been calculated, one or more likelihood scores for different oxidation state combinations for sets for individual ion types forming a given or particular inorganic solid material is/are calculated. And one set of oxidations states for the ion types (i.e., an oxidation state for each ion type) is selected as a function of the one or more likelihood scores (e.g., the highest likelihood score).

In one or more arrangements, one or more of the modules described herein can include artificial or computational intelligence elements, or other machine learning algorithms. Further, in one or more arrangements, one or more of the modules can be distributed among a plurality of the modules described herein. In one or more arrangements, two or more of the modules described herein can be combined into a single module.

Detailed embodiments are disclosed herein. However, it is to be understood that the disclosed embodiments are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various embodiments are shown in FIGS. 1-6C, but the embodiments are not limited to the illustrated structure or application.

The flowcharts and block diagrams in the figures illustrate the architecture, functionality, and operation of possible implementations of systems, methods, and computer program products according to various embodiments. In this regard, each block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. Any kind of processing system or another apparatus adapted for conducting the methods described herein is suited. A typical combination of hardware and software can be a processing system with computer-usable program code that, when being loaded and executed, controls the processing system such that it conducts the methods described herein. The systems, components and/or processes also can be embedded in a computer-readable storage, such as a computer program product or other data programs storage device, readable by a machine, tangibly embodying a program of instructions executable by the machine to perform methods and processes described herein. These elements also can be embedded in an application product which comprises all the features enabling the implementation of the methods described herein and, which when loaded in a processing system, is able to conduct these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a read-only memory (ROM), an erasable programmable read-only memory (EPROM or Flash memory), a portable compact disc read-only memory (CD-ROM), a digital versatile disc (DVD), an optical storage device, a magnetic storage device, or any suitable combination of the foregoing. In the context of this document, a computer-readable storage medium may be any tangible medium that can contain, or store a program for use by or in connection with an instruction execution system, apparatus, or device.

Generally, modules as used herein include routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an application-specific integrated circuit (ASIC), a hardware component of a system on a chip (SoC), as a programmable logic array (PLA), or as another suitable hardware component that is embedded with a defined configuration set (e.g., instructions) for performing the disclosed functions.

Program code embodied on a computer-readable medium may be transmitted using any appropriate medium, including but not limited to wireless, wireline, optical fiber, cable, RF, etc., or any suitable combination of the foregoing. Computer program code for conducting operations for aspects of the present arrangements may be written in any combination of one or more programming languages, including an object-oriented programming language such as Java™, Smalltalk, C++, or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The terms “a” and “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The phrase “at least one of . . . and” as used herein refers to and encompasses any and all possible combinations of one or more of the associated listed items. As an example, the phrase “at least one of A, B, and C” includes A only, B only, C only, or any combination thereof (e.g., AB, AC, BC, or ABC).

Aspects herein can be embodied in other forms without departing from the spirit or essential attributes thereof. Accordingly, reference should be made to the following claims, rather than to the foregoing specification, as indicating the scope hereof.

The preceding description is merely illustrative in nature and is in no way intended to limit the disclosure, its application, or uses. Work of the presently named inventors, to the extent it may be described in the background section, as well as aspects of the description that may not otherwise qualify as prior art at the time of filing, are neither expressly nor impliedly admitted as prior art against the present technology.

As used herein, the phrase at least one of A, B, and C should be construed to mean a logical (A or B or C), using a non-exclusive logical “or.” It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure and are not intended to limit the disclosure of the technology or any aspect thereof. The recitation of multiple variations or forms having stated features is not intended to exclude other variations or forms having additional features, or other variations or forms incorporating different combinations of the stated features.

As used herein the term “about” when related to numerical values herein refers to known commercial and/or experimental measurement variations or tolerances for the referenced quantity. In some variations, such known commercial and/or experimental measurement tolerances are +/−10% of the measured value, while in other variations such known commercial and/or experimental measurement tolerances are +/−5% of the measured value, while in still other variations such known commercial and/or experimental measurement tolerances are +/−2.5% of the measured value. And in at least one variation, such known commercial and/or experimental measurement tolerances are +/−1% of the measured value.

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that a form or variation can or may comprise certain elements or features does not exclude other forms or variations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one variation, or various variations means that a particular feature, structure, or characteristic described in connection with a form or variation or particular system is included in at least one variation or form. The appearances of the phrase “in one variation” (or variations thereof) are not necessarily referring to the same variation or form. It should be also understood that the various method steps discussed herein do not have to be conducted in the same order as depicted, and not each method step is required in each variation or form.

The foregoing description of the forms and variations has been provided for purposes of illustration and description. It is not intended to be exhaustive or to limit the disclosure. Individual elements or features of a particular form or variation are generally not limited to that particular form or variation, but, where applicable, are interchangeable and can be used in a selected form or variation, even if not specifically shown or described. The same may also be varied in many ways. Such variations should not be regarded as a departure from the disclosure, and all such modifications are intended to be included within the scope of the disclosure.

What is claimed is:

1. A system comprising:
a processor; and
a memory communicably coupled to the processor and storing machine-readable instructions that, when executed by the processor, cause the processor to:
calculate joint probability functions for electronic chemical potential and oxidation states for ion types of a solid inorganic material;
calculate a likelihood score for a plurality of oxidation state sets for the ion types in the solid inorganic material; and
select one set of oxidation state from the plurality of oxidation state sets for the ion types as a function of the likelihood score.

2. The system according to claim 1, wherein a joint probability function for an ion type ‘m’ with an oxidation state ‘sⱼ’ (m^{s}^{ⱼ}) is defined by the expression:
Pₘ(μₑ,sⱼ|B)
where μₑ is the electronic chemical potential in the solid inorganic material and B represents boundaries between the oxidation states.

3. The system according to claim 1, wherein the likelihood score is defined by the expression:
$$P ( {μ}_{e} , I \vert \overline{B} ) = {\prod }_{{M}^{{s}_{j}} ∈ I} {P}_{m} ( {μ}_{e} , {s}_{j} \vert \overline{B} )$$
where μₑ is the electronic chemical potential of the solid inorganic material, I is a set of ions in the solid inorganic material, B̅ is the midpoint of boundaries for a given oxidation set as a function of μₑ, sⱼ represents the ‘j^{th}’ an oxidation state, and M^{s}^{ⱼ} represents an element or molecular species with the sⱼ oxidation state.

4. The system according to claim 3, wherein the most likely electronic chemical potential for the solid inorganic material corresponds to the likelihood score with a highest value.

5. The system according to claim 4, wherein a calculated probability function of an oxidation state for each of the ion types forming the solid inorganic material is calculated at a single electronic chemical potential for the solid inorganic material.

6. The system according to claim 1, wherein the memory communicably coupled to the processor and storing machine-readable instructions that, when executed by the processor, cause the processor to train a machine learning module configured to calculate the joint probability functions of the oxidation states for the ion types forming the solid inorganic material.

7. The system according to claim 6, wherein the memory communicably coupled to the processor and storing machine-readable instructions that, when executed by the processor, cause the processor to train the machine learning module with a training data set comprising experimentally determined integer, non-zero oxidation states for ion types of inorganic materials with charge neutrality.

8. The system according to claim 7, wherein the memory communicably coupled to the processor and storing machine-readable instructions that, when executed by the processor, cause the processor to calculate a global instability index for each of the materials in the training data set, the global instability index being a function of a difference between bond valence sums and a nominal oxidation state for each of the ion types forming the inorganic materials.

9. The system according to claim 1, wherein a joint probability density for an ion type ‘m’ with an oxidation state ‘sⱼ’ (m^{s}^{ⱼ}) is defined by the expression:
Pₘ(μₑ,sⱼ|B)
and the likelihood score is defined by the expression:
$$P ( {μ}_{e} , I \vert \overline{B} ) = {\prod }_{{m}^{{s}_{j}} ∈ I} {P}_{m} ( {μ}_{e} , {s}_{j} \vert \overline{B} )$$
where m^{s}^{ⱼ} represents an element or molecular species with the sⱼ oxidation state, μₑ is the electronic chemical potential of the solid inorganic material, B represents boundaries between the oxidation states, I is a set of ions in the solid inorganic material, B̅ is the midpoint of the boundaries for a given oxidation set as a function of μₑ.

10. A system comprising:
a processor; and
a memory communicably coupled to the processor and storing machine-readable instructions that, when executed by the processor, cause the processor to:
train a machine learning module configured to calculate joint probability functions for electronic chemical potential and oxidation states for ion types of a solid inorganic material;
calculate the joint probability functions for electronic chemical potential and oxidation states for ion types of a solid inorganic material;
calculate a likelihood score for a plurality of oxidation state sets for the ion types in the solid inorganic material; and
select one set of oxidation state from the plurality of oxidation state sets for the ion types as a function of the likelihood score.

11. The system according to claim 10, wherein a joint probability density for an ion type ‘m’ with an oxidation state ‘sⱼ’ (m^{s}^{ⱼ}) is defined by the expression:
Pₘ(μₑ,sⱼ|B)
where μₑ is the electronic chemical potential of the solid inorganic material and B represents boundaries between the oxidation states.

12. The system according to claim 10, wherein the likelihood score is defined by the expression:
$$P ( {μ}_{e} , I \vert \overline{B} ) = {\prod }_{{m}^{{s}_{j}} ∈ I} {P}_{m} ( {μ}_{e} , {s}_{j} \vert \overline{B} )$$
where μₑ is the electronic chemical potential of the solid inorganic material, I is a set of ions in the solid inorganic material, B̅ is the midpoint of boundaries for a given oxidation set as a function of μₑ, sⱼ represents the ‘j^{th}’ an oxidation state, and m^{s}^{ⱼ} represents an element or molecular species with the sⱼ oxidation state.

13. The system according to claim 12, wherein the most likely electronic chemical potential for the solid inorganic material corresponds to the likelihood score with a highest value.

14. The system according to claim 13, wherein a calculated probability function of an oxidation state for each of the ion types forming the solid inorganic material is calculated at a single electronic chemical potential for the solid inorganic material.

15. The system according to claim 10, wherein a joint probability density for an ion type ‘m’ with an oxidation state ‘sⱼ’ (m^{s}^{ⱼ}) is defined by the expression:
Pₘ(μₑ,sⱼ|B)
and the likelihood score is defined by the expression:
$$P ( {μ}_{e} , I \vert \overline{B} ) = {\prod }_{{m}^{{s}_{j}} ∈ i} {P}_{m} ( {μ}_{e} , {s}_{j} \vert \overline{B} )$$
where m^{s}^{ⱼ} represents an element or molecular species with the s oxidation state, Je is the electronic chemical potential of the solid inorganic material, B represents boundaries between the oxidation states, I is a set of ions in the solid inorganic material, B̅ is the midpoint of the boundaries for a given oxidation set as a function of μₑ.

16. The system according to claim 10, wherein the memory communicably coupled to the processor and storing machine-readable instructions that, when executed by the processor, cause the processor to train the machine learning module with a training data set comprising experimentally determined integer, non-zero oxidation states for ion types of inorganic materials with charge neutrality.

17. A system comprising:
a processor; and
a memory communicably coupled to the processor and storing machine-readable instructions that, when executed by the processor, cause the processor to:
train a machine learning module configured to calculate joint probability functions for electronic chemical potential and oxidation states for ion types of a solid inorganic material;
calculate the joint probability functions for electronic chemical potential and oxidation states for ion types of a solid inorganic material using the expression, wherein a joint probability density for an ion type ‘m’ with an oxidation state ‘sⱼ’ (m^{s}^{ⱼ}) is defined by the expression:
Pₘ(μₑ,sⱼ|B)
where μₑ is the electronic chemical potential in the solid inorganic material and B represents boundaries between the oxidation states;
calculate a likelihood score for a plurality of oxidation state sets for the ion types in the solid inorganic material; and
select one set of oxidation state from the plurality of oxidation state sets for the ion types as a function of the likelihood score.

18. The system according to claim 17, wherein the likelihood score is defined by the expression:
$$P ( {μ}_{e} , I \vert \overline{B} ) = {\prod }_{{m}^{{s}_{j}} ∈ I} {P}_{m} ( {μ}_{e} , {s}_{j} \vert \overline{B} )$$
where μₑ is the electronic chemical potential of the solid inorganic material, I is a set of ions in the solid inorganic material, B̅ is the midpoint of boundaries for a given oxidation set as a function of μₑ, sⱼ represents the ‘j^{th}’ an oxidation state, and m^{s}^{ⱼ} represents an element or molecular species with the sⱼ oxidation state.

19. The system according to claim 18, wherein the most likely electronic chemical potential for the solid inorganic material corresponds to the likelihood score with a highest value.

20. The system according to claim 19, wherein a calculated probability function of an oxidation state for each of the ion types forming the solid inorganic material is calculated at a single electronic chemical potential for the solid inorganic material.
